(19) United States
(12) Patent Application Publication
(10) Pub. No.: US 2012/0150143 A1
(43) Pub. Date: Jun. 14, 2012

(54) Infusion monitoring device, system and method

(76) Inventors: Kevin Durand, Harvard, MA (US); Charles Grinnell, Groton, MA (US)

(21) Appl. No.: 13/373,781

(22) Filed: Nov. 30, 2011

Related U.S. Application Data
(62) Division of application No. 11/436,110, filed on May 17, 2006, now abandoned.
(60) Provisional application No. 60/682,527, filed on May 17, 2005.

(51) Int. Cl. A61M 5/168

(52) U.S. Cl. 604/500; 604/67; 604/66

(57) ABSTRACT

A system for automatically monitoring an infusion in a patient receiving an infusion from a fluid source, the system including: an infusion monitoring device comprising: a fluid valve, disposed in a fluid connection between the fluid source and the patient, and operable for preventing or allowing the flow of fluid from the fluid source to the patient; a variable volume chamber, fluidly coupled between the fluid valve and the patient, for allowing the creation of a pressure differential between the variable volume chamber and the patient; and a fluid inspection window, disposed in the fluid connection between the variable volume chamber and the patient, for allowing a view of fluid drawn from the patient into the infusion monitoring device when the fluid valve is closed and the variable volume chamber creates a negative pressure differential between the variable volume chamber and the patient.

### RELATED APPLICATION

This application is a divisional of prior U.S. patent application Ser. No. 11/436,110, filed May 17, 2006, which is related to and claims priority from U.S. Provisional Application No. 60/682,527, filed May 17, 2005, entitled Intravenous Detection System which are both incorporated fully herein by reference.

### TECHNICAL FIELD

The present invention relates to the infusion of fluid into a patient, and more particularly relates to a device, system and method for administration of infusions and infusion monitoring.

### BACKGROUND INFORMATION

Infusion is the administration of a fluid-based substance into a patient, most typically this is performed directly into the venous side of the patient's vascular system but can also be performed into the arterial side of the patient's vascular system or into the subcutaneous tissue and not directly into the vascular system. For purposes of the description of the present invention, infusion shall mean any or all of the possible methods of administering fluid-based substances into a patient. The variety of aspects and embodiments of the present invention apply to all forms of infusion.

The simplest form of infusion is a syringe coupled to a hollow needle or catheter, commonly referred to as an injection. The needle is inserted through the skin into a vein, and the contents of the syringe are injected through the needle into the bloodstream. Generally, the most accessible veins are the metacarpal veins in the forearm of a patient. Typically, a tourniquet is applied to restrict the flow of blood, thus causing the veins to bulge. The needle is then inserted through the skin and into the vein and the tourniquet is removed. After the needle is inserted, it is common to draw back slightly on the syringe to see a blood return, thus verifying that the needle is within the vein and not the surrounding tissue.

Some syringes, as shown in FIG. 1, have been designed to provide a visual blood spot. A pre-marked transparent section of the syringe allows the administrator to view the blood drawn into the syringe prior to injection of the fluid contents of the syringe. The specific example in FIG. 1 is a Wyeth aspiration syringe. The Wyeth aspiration syringe provides a hollow tube within the body of the syringe. Prior to the injection of fluid from the syringe, the administrator is provided with visual confirmation that the tip of the needle is within the patient's vein. This device, however, as well as others like it, are manually operated and cannot connect to or interact with other devices or systems of fluid delivery or problem reporting.

Infusion can be intermittent or continuous; continuous administration is most often performed directly into one of the patient's veins and is referred to as an intravenous infusion. An intravenous infusion consists of a needle or a catheter, inserted through the skin into a venous system commonly, but not always, into a peripheral vein. A peripheral vein is any vein that is not in the chest or abdomen. Part of the needle or catheter remains outside the skin, with a hub that can be connected to a syringe or an intravenous infusion line, or capped. An infusion pump may be used to provide precise control over the flow rate and total amount passed through the infusion line.

Various adverse effects can be caused by improper location of the needle or catheter within the vein or surrounding tissue, as shown in FIG. 2. Infiltration occurs when the tip of the needle or catheter withdraws from the vein or penetrates through the vein into surrounding tissue. Infiltration occurs frequently with continuous infusion. The symptoms of infiltration may include bruising, swelling, minor discomfort to moderate pain, and temporary limited limb use. Infiltration is normally associated with the injection of non-vesicant compounds, for example, normal saline, antibiotics in dilution, and Ringer's lactate. Injection of irritating medication, for example valium, total parenteral nutrition and chemotherapy compounds may intensify the effects caused by infiltration and cause extravasation. Extravasation is the inadvertent administration of a vesicant substance into the surrounding tissues. Extravasation can have disastrous outcomes with symptoms ranging from severe swelling, burning, severe pain, tissue erosion and necrosis. Many follow-up treatments may be required to cure the effects of extravasation. Adverse effects in infusion cause additional treatments, ranging from antidote administration to extensive tissue and skin repair to limb amputations.

Regular monitoring of infusion sites helps reduce the severity of adverse effects when infiltration and extravasation occurs. Based on the foregoing, it is apparent that there is a need for an automated device, system, and method for administration of infusions that provide regular monitoring of infusions.

### SUMMARY OF THE INVENTION

The present invention provides a device, system, and method for monitoring infusion of fluids into a patient. The present invention may be used for either intermittent or continuous, manual or automatic infusion monitoring. The present invention may also be used while infusing a variety of medications and fluids, both vesicant and non-vesicant compounds, into a patient. The present invention may have an integral needle or catheter for infusing fluids into the patient, may operate with the addition of a standard needle or catheter familiar to those skilled in the art, or may be integrated into and/or with components forming a larger more comprehensive system.

One or more sources of fluid may connect to the proximal end of the infusion monitoring device and allow gravity “drip” type of intravenous delivery or a mechanized “pump” infusion type of delivery. The fluid flows from the source through the infusion monitoring device through the needle or catheter into the patient.

The infusion monitoring device may include a pad or other similar “region” or “features” for securing the infusion monitoring device to the patient. One skilled in the art will appreciate that this can be provided by various means such as having a self-adhesive area for allowing the infusion monitoring device to be adhered to the skin of a patient to maintain the device in place.

The infusion monitoring device also includes a variable volume chamber, which may exist in one or more forms, any or all of which will be collectively referred to herein as a variable volume chamber. The primary purpose of the variable volume chamber is to provide a method to create a negative pressure differential between the infusion monitoring device and the patient so that fluid will be drawn from the patient at the infusion site and into the infusion monitoring device for observation. The variable volume chamber is in fluidic connection with the needle or catheter. The variable volume chamber is designed and constructed such that it's volume can be increased or decreased which allows the volume of fluid contained in the intravenous injection monitoring device to increase or decrease. In one embodiment, the volume or capacity of the variable volume chamber is typically sized to match the size and volume of the catheter/needle and other fluid coupling between the variable volume chamber and the patient.

The infusion monitoring device also contains a fluid valve to interrupt and resume the flow of fluid between the infusion fluid supply and the patient and is placed between the fluid source and the variable volume chamber. One skilled in the art will appreciate that the fluid valve may be accomplished by a wide variety of means common in devices that control the flow of medical and other fluids. One skilled in the art will also appreciate that the valve may be normally closed or normally open, and may or may not require power to stay in either the open or closed position.

An optional actuator may be located in the infusion monitoring device and coupled to the variable volume chamber, for causing the variable volume chamber to expand thereby creating a negative pressure or negative pressure gradient in the injection fluid line coupled to the patient, or contract creating a positive pressure or positive pressure gradient in the injection fluid line coupled to the patient. With a fluid valve closing the path between the infusion monitoring device and the fluid supply, a negative pressure or negative pressure gradient creates a tendency for fluid from the patient to be drawn proximally through the needle or catheter into the infusion monitoring device. One skilled in the art will appreciate that the actuator may derive its motive power from screws, drive wheels, levers, pneumatic connections, fluid connections, gravity, or direct human touch, any or all of which may cause the variable volume to expand or contract. The variable volume chamber may also be actively actuated to change its volume, and then return to its original volume without active actuation. The volume of fluid in the variable volume chamber may also change as a result of the pressure in the venous system of the patient either in combination with an actuator or without the need for an actuator, as well as with or without the need for a vent.

A fluid inspection window is typically located distally to the variable volume chamber that is, between the variable volume chamber and the patient, in order to view and monitor the injection fluid within the infusion monitoring device. The window may be a clear component, positioned to allow the administrator to directly, visually view and monitor the fluid within the infusion monitoring device. Alternatively or in addition, a sensor may either replace the window or be located in proximity to the window, for sensing the presence, composition or characteristics of the fluid within the infusion monitoring device. One skilled in the art will appreciate that a variety of sensors or sensor type devices may be used to determine various conditions such as the presence, absence, composition, characteristic, flow rate or even temperature of the fluid. Such sensors include, but are not limited to ultrasonic sensors, light sensors, optical sensors, and cameras or probes of various types. The sensors or sensor type devices may perform their function with or without contacting the fluid. The sensing may also occur with the addition of a chemical marker or reagent that is added to the fluid and may or may not be removed from the infusion monitoring device for further processing.

The infusion monitoring device, system and method of the present invention allows the administrator to periodically monitor the administration of a fluid based substance to determine that the needle or catheter is in the correct position within the patient. If the distal end of the needle or catheter is not in a vein, when the infusion monitoring device draws fluid from the patient, no blood will be present in the window. The presence or absence of blood in the window may or may not be desirable depending on where the needle or catheter is supposed to be located in the patient.

The presence, composition or characteristics of the fluid in the window when the infusion monitoring device draws fluid from the patient may be viewed directly by an administrator who is present, or by a sensor that provides a signal or alarm. The signal or alarm provided by the sensor may be communicated directly by the infusion monitoring device in the form of any combination of visual, auditory or electronic indicators or remotely to one or more devices that provide any combination of visual, auditory or electronic indicators that may be present at or near the patient, or may be present at a remote location such as a nursing station. The signal or alarm provided by the sensor may be communicated from the infusion monitoring system remotely through a wire or by a wireless connection.

The infusion monitoring system may or may not provide for programming various functions or functionalities of the infusion monitoring device. Examples of user programmability include, but are not limited to: the inspection times, intervals or periods; alarm trigger thresholds; what is to be recorded; how long is recorded information to be kept; reporting format, criterion and frequency; and acceptable range of sensor values. The infusion monitoring device may or may not control, or cause to limit, or allow for the setting of limits on the volume of fluid drawn from the patient as well as setting limits on the pressure created to draw fluid from, or deliver fluid to the patient.

The infusion monitoring system may or may not have the capability to provide a back and forth fluid flow (reciprocating cycle) at the distal end of the needle or catheter. This flow can be used to prevent blood from clotting at or near the end of the needle or catheter that would occlude the fluid pathway to the patient.

### EMBODIMENTS OF THE INVENTION

The present invention contemplates several embodiments of the infusion monitoring device and system of the present invention.

In the first embodiment of the present invention, the infusion monitoring device is a simple manual device to expedite a manual, visual check. A single or multiple user actions may occlude the line and attempt to draw fluid from the patient for visual inspection.

In another embodiment of the present invention, which may be entitled the basic automated device, a battery powered infusion monitoring system checks for the presence, composition or characteristics of the fluid at a predetermined interval and sounds an alarm and/or occludes the line if an infiltration or other anomalous condition exists, without remote connections. The goal of this device is to add automation to the task in the most simple way possible. This method may or may not allow manual inspections (manual override or one-touch operation). It is contemplated that this method of operation could include a range of devices that may be preset for different inspection intervals, pressures, volumes, acceptable range of sensor values, etc. This may be able to be done without software, which would greatly reduce the system complexity and regulatory requirements.

In a further embodiment of the present invention, the infusion monitoring device may be operated as a programmable device with user adjustable settings for inspection frequency, fluid pressures, volumes, acceptable range of sensor values, etc.

In yet another embodiment of the present invention, the infusion monitoring system may record and/or report the current state and history of inspections and alarms to the clinician either locally or remotely by wired or wireless transmission.

In a further contemplated embodiment of the present invention, the infusion monitoring device may be integrated with one or more other patient care devices such as those for infusion, patient monitoring, diagnostics or any other purpose. The infusion monitoring device of the present invention may operate cooperatively with these devices by sharing a power source, a user interface, alarm enunciation, control commands, data storage, or the like.

In a further contemplated embodiment of the present invention, the infusion monitoring system may monitor certain characteristics of the infusate such as ensuring that the fluid source is still providing a fluid (bag not empty); that the fluid is the correct fluid; that the fluid rate and/or temperature are as expected, and other such monitoring.

In a further contemplated embodiment of the present invention, the infusion monitoring system may monitor certain properties or characteristics of the patient's blood beyond the mere presence of blood. These properties or characteristics may include blood glucose, oxygen saturation, or any number of other properties or characteristics, that can be evaluated in situ via the window within the primary fluid path. These properties or characteristics may also include any number of other properties or characteristics that must be evaluated external to the primary flow of the device in which case the blood or fluid may be withdrawn and/or re-directed from the fluid path to be analyzed.

In any of the previously mentioned embodiments, all or part or none of the infusion monitoring device may be disposable and intended for a single use while all or part or none of the device may be reused.

Although the present invention is described in connection with one or more exemplary embodiments, any of which may be used alone or combined in whole or in part with other disclosed embodiments, this is not a limitation of the present invention which is not to be limited except by the allowed claims and their legal equivalents.

### BRIEF DESCRIPTION OF THE DRAWINGS

These and other features and advantages of the present invention will be better understood by reading the following detailed description, taken together with the drawings wherein:

FIG. 1 is a top and side profile of a prior art aspiration syringe;

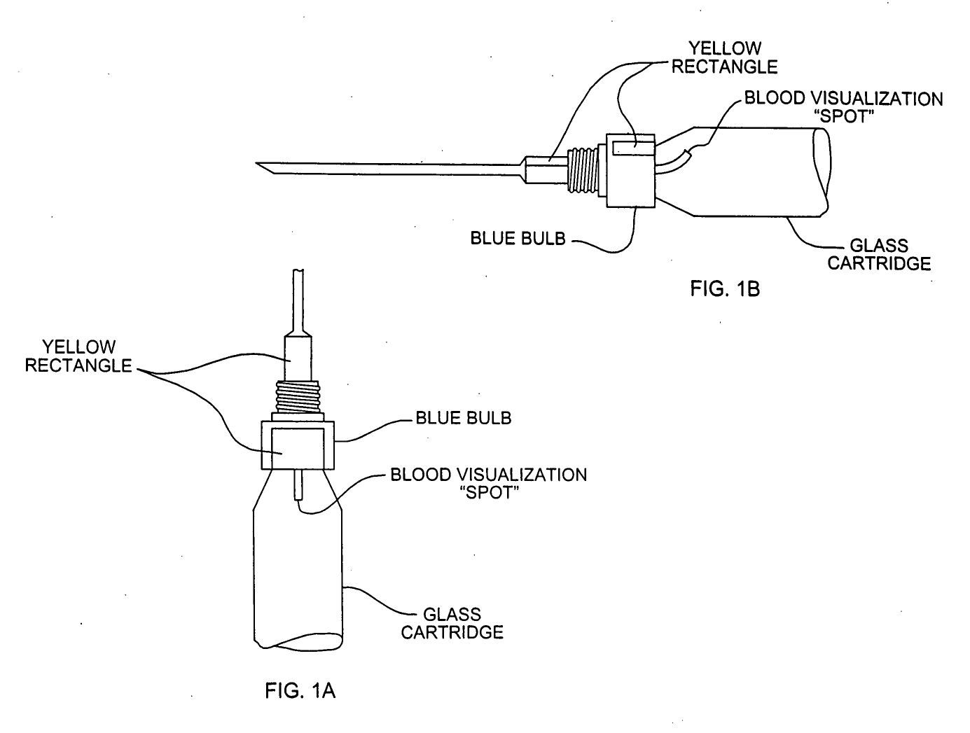

FIG. 2 is a profile view of a dislodged intravenous needle;

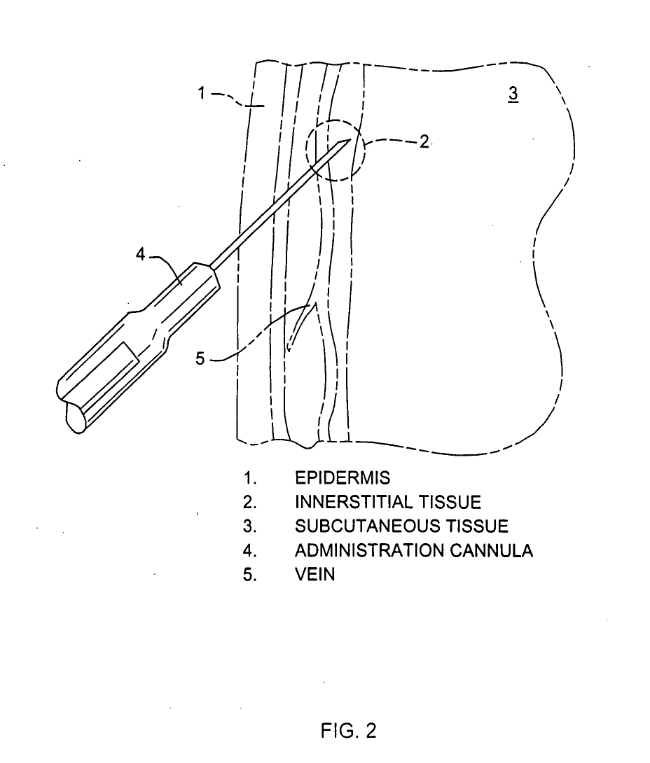

FIG. 3 is a functional schematic block diagram of the infusion monitoring device according to the present invention;

FIG. 4 is a perspective view of one implementation of the infusion monitoring device according to an exemplary embodiment of the present invention shown with and without its outer shell;

FIG. 5 is a perspective view of one implementation of the infusion monitoring device including an attached control device according to an exemplary embodiment of the present invention;

FIG. 6 is a flow chart of an exemplary infiltration and extravasation detection algorithm according to one aspect of the present invention;

FIG. 7 is a flow chart of an exemplary infiltration and extravasation detection-locking algorithm according to another aspect of the present invention; and ,

FIG. 8 is a flow chart of an exemplary KVO (Keep Vessel Open) algorithm according to yet another aspect of the present invention.

### DETAILED DESCRIPTION OF THE DRAWINGS

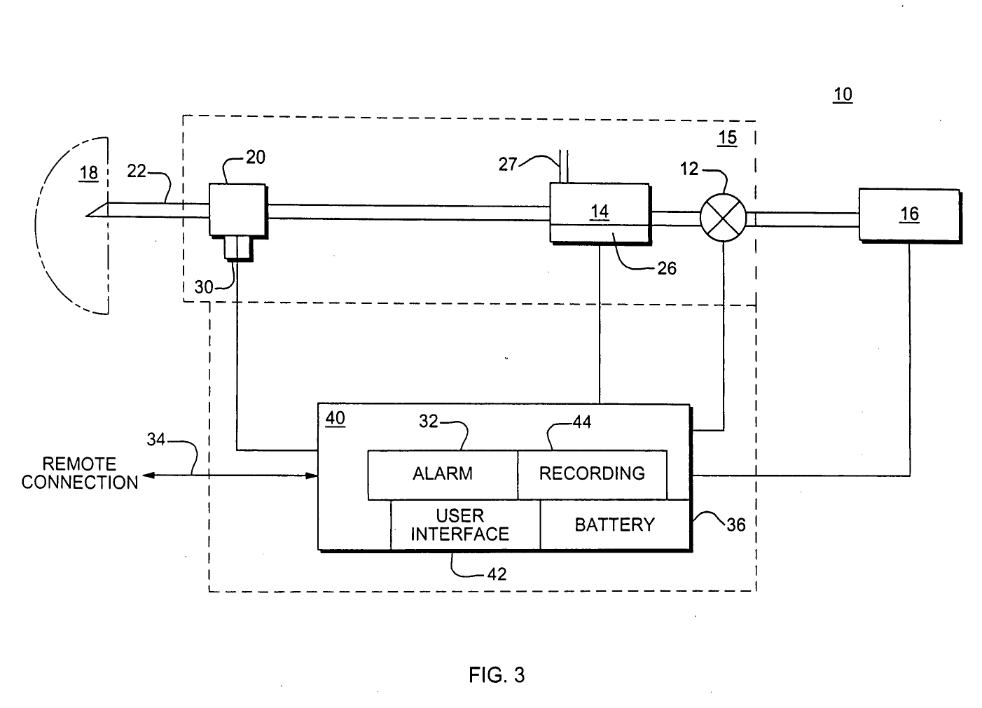

The infusion monitoring device 15, FIG. 3, according to the present invention includes a fluid valve 12 typically located between the variable volume chamber 14, which may exist in one or more forms, coupled to a source of infusion fluid 16. The fluid valve 12 serves to interrupt and resume the flow of fluid from the fluid source 16 when the infusion monitoring device 15 monitors the infusion of fluid, as will be described below.

The infusion monitoring device 15 according to the present invention also includes a window 20 typically located between the variable volume chamber 14 and the patient 18, to allow a user, either directly or with the use of a sensor 30, to determine and/or monitor the presence, properties or characteristics of the fluid within the infusion monitoring device.

During infusion, fluid flows from the infusion supply 16 through the infusion monitoring device 15 through the catheter or needle 22 into the patient 18.

An actuator 26, which may be manual or automatic, may optionally be located in the infusion monitoring device 15 and coupled to the variable volume chamber 14, for causing the variable volume chamber 14 to expand or to contract.

With a fluid valve 12 closing the path between the infusion monitoring device 15 and the injection fluid supply 16, a pressure gradient, caused by expanding the variable volume chamber 14 and/or venous pressure in the patient's system, creates a tendency for fluid from the patient 18 to flow proximally through the needle or catheter 22 into the infusion monitoring device 15 due to the pressure differential in the line between the patient 18 and the infusion monitoring device 10 and with or without the use of a vent 27.

The presence or absence of blood in the window 20 may or may not be desirable depending on where the needle/catheter 22 is supposed to be located in the patient 18. For example, if the needle/catheter 22 is supposed to be in the patient's venous system, then blood should be present in a window 20 when the infusion monitoring device 15 of the present invention draws fluid from the patient. Alternatively, if the needle/catheter 22 is not supposed to be positioned in the patient's venous system, the presence of blood in the window 20 would indicate that the needle/catheter 22 is positioned incorrectly. Those skilled than the art will understand and appreciate how the infusion monitoring system 10 according to the present invention may be utilized as described herein.

The present invention contemplates that the valve 12, variable volume chamber 14, actuator 26 and window 20 may be integrated to form a device hereinafter referred to as the “simple manual device” 15 that may or may not also include a vent 27 and needle or catheter 22. It is further contemplated that the infusion monitoring system 10 of the present invention may include additional elements and functionality to automate the sensing and reporting of the presence, properties, composition or characteristics of the fluid in the infusion monitoring device 15 as will be described below in connection with a system.

In the present invention, the presence, properties or characteristics of the fluid in the infusion monitoring system 10 may be determined by the administrator or by a sensor 30 that provides a signal or alarm 32 to indicate the readings of the sensor and/or the presence of a potential problem.

The infusion monitoring system 10 may be powered by a battery 36 (AC current—not shown) or be powered by some other source via a remote connection 34.

The infusion monitoring system 10 may include a user interface 42 to allow for programming and other information input and output that may be desired. The user interface 42 may be accessed either locally or by wired or wireless transmission 34.

The infusion monitoring system 10 may have the addition of the ability to record 44 and/or report 34 the current state and history of inspections to the clinician either locally or by wired or wireless transmission 34.

The signal or alarm 32 provided by the sensor 30 may be communicated directly by the infusion monitoring device in the form of any combination of visual, auditory or electronic indicators. The signal or alarm 32 provided by the sensor 30 may be communicated from the infusion monitoring device remotely 34 to one or more devices that provide any combination of visual and auditory indicators that may be present at or near the patient, or may be present at a remote location such as a nursing station. The signal or alarm 32 provided by the sensor 30 may be communicated from the infusion monitoring device remotely 34 through a wire or by a wireless transmission.

It is further contemplated that the alarm 32, remote connection 34, battery 36, user interface 42 and recorder 44 may be integrated to form a device 40 hereinafter referred to as the “controller” 40 to connect with or to the simple manual device 15. The controller 40 may include a memory, processor and user inputs and outputs as described herein. It is further contemplated that the controller 40 may be a separate, stand-alone device or may be part of or incorporated with the simple manual device 15 to form the infusion monitoring system 10.

It is further contemplated that the simple manual device 15 and/or controller 40 may be operated in connection with or integrated with other patient care devices such as those for infusion (infusion pumps for example), patient monitoring, diagnostics or any other purpose via remote connection 34.

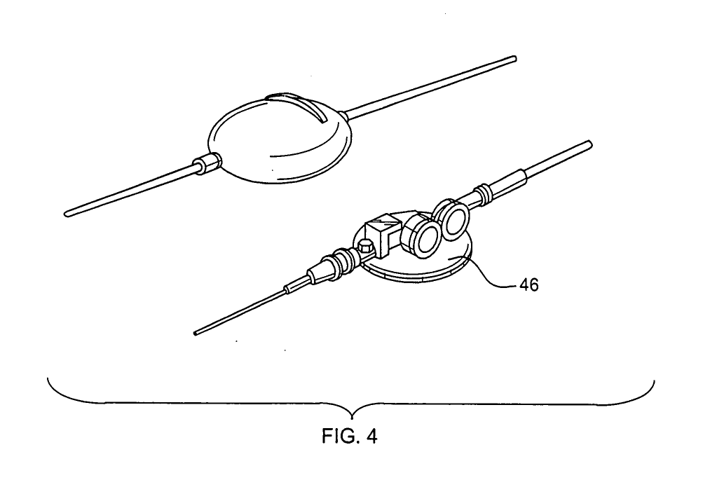

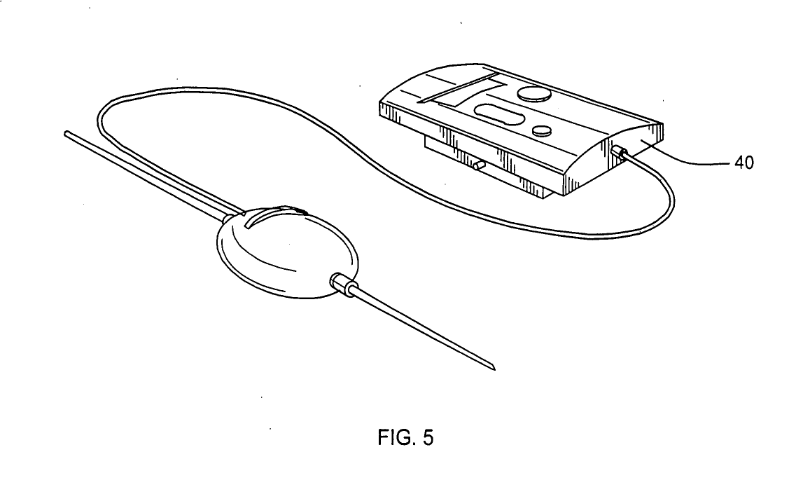

Contemplated physical embodiments of the infusion monitoring device 15 according to the invention are shown in FIGS. 4 and 5. The outer body of the infusion monitoring device 15 may be designed to allow easy handling by the administrator. The infusion monitoring device 15, or portions thereof, may also be designed and constructed to allow for sterilization by a variety of means familiar to those skilled in the art through the choice of appropriate materials, shape, features and/or packaging. The infusion monitoring device 15 may also be designed to be produced inexpensively so that the unit, or portions thereof, may be disposed of after a single use on a patient.

The infusion monitoring device may also provide a shaped or flat adhesive surface 46, FIG. 4, to secure the device to the patient close in proximity to the intravenous access device. The adhesive surface 46 may be rigid or flexible in nature.

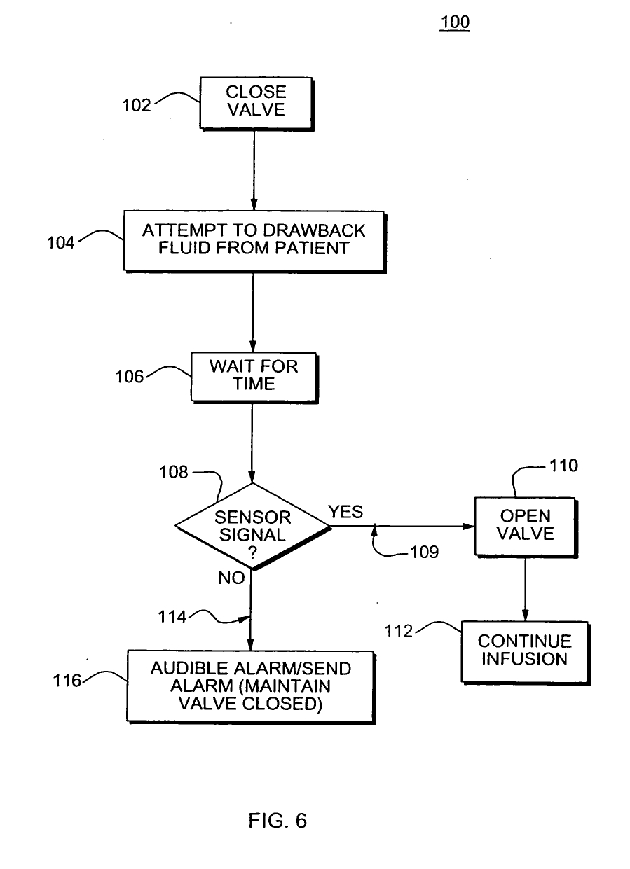

The series of acts 100, FIG. 6 describes the infiltration and extravasation detection method according to another aspect of the present invention. The method according to the present invention determines whether the needle is in the correct position within the patient's vein. The valve 12 shuts off the flow from the infusion supply 16, act 102. The variable volume chamber 14 expands creating a negative pressure that attempts to draw fluid from the patient's vein through the needle or catheter 22, into the infusion monitoring device 15, act 104.

The infusion monitoring system 10 waits for a predetermined or programmed time, act 106.

The next action, 108, determines if there is a signal from the optical sensor determining a change in the fluid in the “window” (i.e. there is or is not blood in the window depending on what the device is looking for), act 108.

If act 108 is “yes”, branch 109 is followed and valve 12 is opened, act 110, and the infusion is continued, act 112. The variable volume chamber 14 returns to its original state and forces fluid back through the needle of catheter 22 into the patient's vein. The infusion of fluid from the supply continues until another specified check of the infusion.

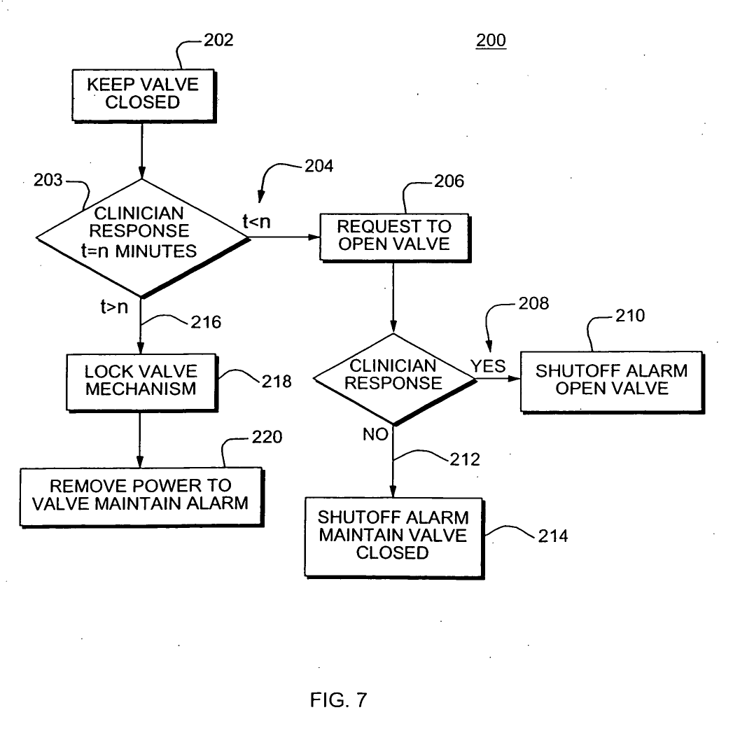

If act 108 determines that there is no signal from the optical sensor or a manual determination of no change in the fluid in the “window” (i.e. there is something other than blood in the window), the “no” branch 114 is taken, and an alarm may be sounded and additional actions may be put into motion, act 116, and may include the occlusion algorithm, FIG. 7.

The occlusion algorithm, FIG. 7, is an exemplary algorithm for maintenance of an occlusion when an infiltration is detected. The infusion monitoring system maintains power to the valve 12 or otherwise keeps the valve closed, act 202. The infusion monitoring system then determines if a clinician has responded within a predetermined time, act 203. If the response time, “t”, is prior to a predetermined time, “n”, act 204, the infusion monitoring system requests, act 206, of the clinician to decide if the device may remove the occlusion. If a clinician responds yes, act 208, the alarm is shut off and the valve is opened, act 210. If the clinician responds no, act 212, the alarm is shut off and occlusion is maintained, act 214. If the clinician does not respond within the predetermined time, act 216, the infusion monitoring system keeps the valve 12 closed, act 218, power is shut off to the valve and the alarm is maintained, 220.

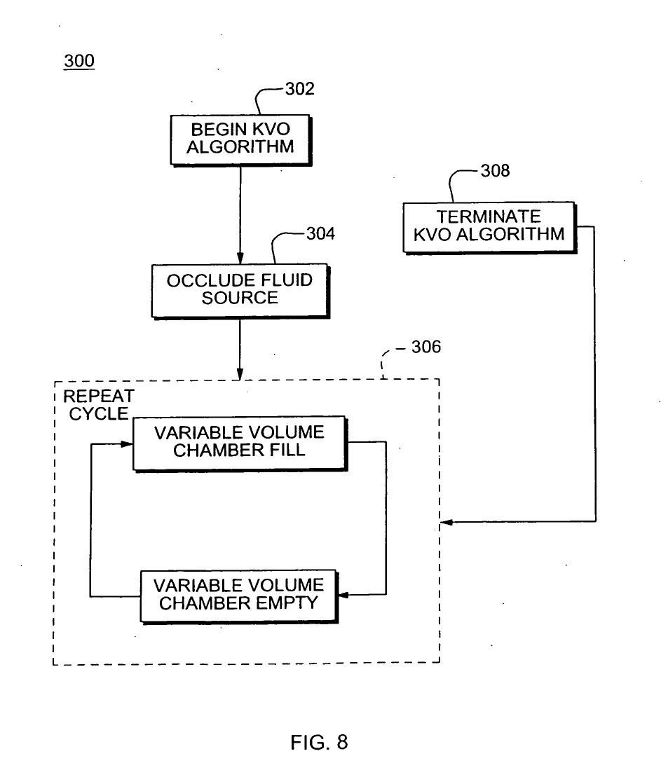

According to another aspect of the present invention, the system may be operated in an embodiment to keep the infusion needle or catheter open (referred to herein as KVO). The KVO algorithm, FIG. 8, is an exemplary algorithm for maintenance of patency of the infusion needle or catheter 22. When commanded to start the KVO algorithm, act 302, the infusion monitoring system closes valve 12, act 304. The infusion monitoring system then repeats a cycle of filling and emptying the variable volume chamber 14, act 306, until commanded to terminate the KVO algorithm, act 308.

Although the present invention has been described in connection with one or more exemplary embodiments, any of which may be used alone or combined in whole or in part with other disclosed embodiments, this is not a limitation of the present invention which is not to be limited except by the allowed claims and their legal equivalents. In addition, modifications and substitutions by one of ordinary skill in the art are considered to be within the scope of the present invention.

1. A system for automatically monitoring an infusion in a patient receiving an infusion from a fluid source, the system comprising: an infusion monitoring device comprising:
a fluid valve, disposed in a fluid connection between said fluid source and said patient, and operable for preventing or allowing the flow of fluid from said fluid source to said patient;
a variable volume chamber, fluidly coupled between said fluid valve and said patient, for allowing the creation of a pressure differential between said variable volume chamber and said patient;
a fluid inspection window, disposed in said fluid connection between said variable volume chamber and said patient, for allowing a view of fluid drawn from said patient into said infusion monitoring device when said fluid valve is closed and said variable volume chamber creates a negative pressure differential between said variable volume chamber and said patient;
at least one sensor, disposed proximate with said fluid inspection window, for facilitating automated inspection of said fluid in said fluid inspection window; and
a controller, coupled to said fluid valve and to said at least one sensor, for controlling operation of said fluid valve between an open and a closed position and responsive to said sensor, for performing automated inspection of said fluid in said fluid inspection window and determining the status of said infusion.

2. The system of claim 1 wherein said controller causes an alarm to sound if said infusion is determined to be faulty.

3. The system of claim 2 wherein said alarm is sounded locally proximate said patient.

4. The system of claim 2 wherein said alarm is sounded remote from said patient.

5. The system of claim 2 wherein said controller records the current state and/or history of infusion inspection data and alarms.

6. The system of claim 5 wherein said controller reports said recorded data.

7. The system of claim 1 wherein said controller is programmable.

8. The system of claim 1 wherein said sensor is selected from the group consisting of ultrasonic sensors, light sensors, optical sensors, cameras and probes.

9. The system of claim 1 wherein said sensor is adapted to detect one or more conditions selected from the group consisting of: presence or absence of blood in said fluid inspection window, composition and/or characteristics of patient's blood, and composition and/or characteristics of fluid being infused.

10. The system of claim 1 wherein a sample of said fluid drawn from said patient is removed from the infusion monitoring device and processed with or without diluent, reagent, incubation enhancer or other standard sample preparation methods.

11. The system of claim 1 wherein the infusion monitoring device is integrated with one or more other patient care devices selected from the group consisting of: infusion devices, patient monitoring devices, and diagnostics devices.

12. The system of claim 11 wherein the infusion monitoring device operates cooperatively with said integrated patient care device by sharing one or more functions from the group consisting of: power source, user interface, alarm enunciation, control commands, data storage, and communications capabilities.

13. A method for monitoring an infusion in a patient, said method comprising the acts of: providing an infusion monitoring device, said infusion monitoring device including a fluid inspection window disposed between the fluid valve and the patient; and monitoring said fluid in said fluid inspection window of said infusion monitoring device.

14. The method of claim 13 wherein monitoring said fluid in said fluid inspection window includes monitoring the fluid being infused in said patient for one or more characteristics.

15. The method of claim 13 wherein a fluid valve is disposed in line with said fluid inspection window for the purpose of stopping the flow of infusion fluid as needed for inspection.

16. A method for keeping a patient infusion open comprising the acts of:
providing an infusion monitoring device, said infusion monitoring device including at least: a fluid valve, disposed in a fluid connection between a fluid source and a patient, and operable for preventing or allowing the flow of fluid from said fluid source to said patient and a variable volume chamber, fluidly coupled between said fluid valve and said patient, for allowing the creation of a negative pressure differential between said variable volume chamber and said patient tending to draw fluid from the patient into the variable volume chamber and for allowing the creation of a positive pressure differential between said variable volume chamber and said patient tending to push fluid back from said variable volume chamber to said patient;
closing said fluid valve; creating a negative pressure differential between said variable volume chamber and said patient causing fluid from said patient at an infusion site to be drawn into said variable volume chamber of said infusion monitoring device;
creating a positive pressure differential between said variable volume chamber and said patient causing fluid from variable volume chamber to be pushed into said patient at said infusion site; and
repeating the cycle of drawing fluid from and pushing fluid to the patient.

17. A system for automatically monitoring an infusion in a patient receiving an infusion from a fluid source, the system comprising: an infusion monitoring device comprising:
a fluid valve, disposed in a fluid connection between said fluid source and said patient, and operable for preventing or allowing the flow of fluid from said fluid source to said patient;
a variable volume chamber, fluidly coupled between said fluid valve and said patient, for allowing the creation of a pressure differential between said variable volume chamber and said patient; and
a fluid inspection window, disposed in said fluid connection between said variable volume chamber and said patient, for allowing a view of fluid drawn from said patient into said infusion monitoring device when said fluid valve is closed and said variable volume chamber creates a negative pressure differential between said variable volume chamber and said patient.

18. The system of claim 17, further including at least one sensor, disposed proximate with said fluid inspection window, for facilitating automated inspection of said fluid in said fluid inspection window.

19. The system of claim 18, further including a controller, coupled to said fluid valve and to said at least one sensor, for controlling operation of said fluid valve between an open and a closed position and responsive to said sensor, for performing automated inspection of said fluid in said fluid inspection window and determining the status of said infusion.
